(19) United States
(12) Patent Application Publication
(10) Pub. No.: US 2025/0054140 A1
(43) Pub. Date: Feb. 13, 2025

(54) METHOD FOR DETERMINING A MEDICAL IMAGE DATA SET AND PROVISION SYSTEM

(71) Applicant: Siemens Healthineers AG, Forchheim (DE)

(72) Inventor: Tom WEIDNER, Erlangen (DE)

(73) Assignee: Siemens Healthineers AG, Forchheim (DE)

(21) Appl. No.: 18/794,258

(22) Filed: Aug. 5, 2024

(30) Foreign Application Priority Data
Aug. 8, 2023 (DE) 10 2023 207 609.8

(51) Int. Cl. G06T 7/00; G06T 11/00; G16H 30/20; G16H 30/40

(52) U.S. Cl. CPC G06T 7/0012; G06T 11/003; G16H 30/20; G16H 30/40; G06T 2207/30068; G06T 2207/30096

(57) ABSTRACT

One or more example embodiments relates to a computer-implemented method for determining a medical image data set from raw data using a medical imaging facility. The method comprises reconstructing a preliminary image data set from the raw data via a first reconstruction facility, wherein reconstruction parameters of a predetermined default parameter set are used; analyzing the preliminary image data set via an analysis algorithm on an analysis facility to determine an item of analysis information that describes image content of the preliminary image data set; determining a second parameter set of reconstruction parameters as a function of the analysis information; and determining the medical image data set from at least one of the raw data or the preliminary image data set via the first reconstruction facility or a further reconstruction facility, wherein the reconstruction parameters of the second parameter set are used to determine the medical image data set.

### CROSS-REFERENCE TO RELATED APPLICATION(S)

The present application claims priority under 35 U.S.C. § 119 to German Patent Application No. 10 2023 207 609.8, filed Aug. 8, 2023, the entire contents of which is incorporated herein by reference.

### FIELD

One or more example embodiments relates to a computer-implemented method for determining a medical image data set, which is to be analyzed by an evaluator and/or which is to be archived, from raw data of an acquisition process using a medical imaging facility. In addition, one or more example embodiments relates to a provision system and a computer program product.

### RELATED ART

Medical imaging procedures are becoming increasingly important in medical diagnostics. In the case of many imaging modalities, such as magnetic resonance imaging, computed tomography/tomosynthesis and positron emission tomography (PET), raw data, such as k-space data, projection images and correlating events, is first acquired, from which medical image data sets that are actually to be analyzed for diagnostic purposes are then reconstructed. On the other hand, due to the number of patients to be examined, a high throughput must be achieved, which means that the total duration of the examination using the medical imaging facility should not be too long. In many radiology practices and clinics, the actual acquisition process of the raw data on the medical imaging facility is only monitored by a medical technical assistant (MTA), while the radiologist is not present. The MTA assesses at the imaging facility itself whether the acquisition process was successful, usually on a (first) reconstruction. Reconstructed medical image data sets are then forwarded to a diagnostic workstation or stored in an image archiving facility, such as a PACS, for retrieval from the diagnostic workstation, where the radiologist carries out the evaluation (diagnosis) based on the medical image data set.

Particularly in the case of screening examinations or other initial examinations without a specific anamnesis background that already indicates an existing abnormality, it is difficult to adapt the reconstruction or determination of the medical image data set parameterized by a large number of reconstruction parameters to individual patients, so that a standard reconstruction parameter set is usually provided for such cases. Especially in screening examinations, for example in mammography, it is not known in advance which are the most relevant structures in the tissue. On the other hand, it would be important for further examinations and/or treatment to draw the best possible conclusions from the result of the acquisition process.

For this purpose, it is only known in the prior art that a radiologist can suggest a new, adapted reconstruction, for which it is then to be hoped that the raw data, which usually represents a large amount of data that is not archived, is still available, for example at the medical imaging facility. This takes additional time, but the medical image data set, which is the result of the new reconstruction, must also be archived. This, as well as the longer or permanent storage of raw data, runs counter to the desire to minimize transmission times and storage space for large image data sets, such as those that occur in three-dimensional applications and/or tomosynthesis applications. Each new reconstruction exacerbates this problem, especially if raw data actually has to be stored for longer.

In addition, new reconstructions or additional processing steps usually have to be carried out manually on the reconstruction facility, which is usually part of the medical imaging facility (reimporting the raw data, reprocessing, resending), which may even take place iteratively. Re-reconstruction should therefore be avoided where possible, as it is time-consuming and can interrupt both the diagnostic workflow and the acquisition workflow.

It is known for many medical imaging facilities to provide multiple standard parameter sets of reconstruction parameters, for example for special situations and/or workflows such as biopsy, magnification, patients with implants and the like. Such standard parameter sets can also be customized, for example to meet the requirements of a larger number of radiologists.

### SUMMARY

One or more example embodiments provides a way of obtaining patient-specific medical image data sets quickly and, in particular, automatically, which are suitable for improved evaluation.

### BRIEF DESCRIPTION OF THE DRAWINGS

Further advantages and details are apparent from the exemplary embodiments described below and with the aid of the drawings. In the drawings:

FIG. 1 shows a first embodiment of a provision system according to the invention,

FIG. 2 shows a flow chart for explaining a first exemplary embodiment of the method according to the invention,

FIG. 3 shows a second embodiment of a provision system according to the invention, and

FIG. 4 shows a flow chart for explaining a second exemplary embodiment of the method according to the invention.

### DETAILED DESCRIPTION

According to one or more example embodiments, a method of the type mentioned in the introduction has the following steps:
reconstructing a preliminary image data set from the raw data via a first reconstruction facility, wherein reconstruction parameters of a predetermined default parameter set are used,
analyzing the preliminary image data set via an analysis algorithm on the analysis facility to determine an item of analysis information that describes the image content,
determining a second parameter set of reconstruction parameters as a function of the analysis information, and
determining the medical image data set from the raw data and/or the preliminary image data set via the first reconstruction facility or a further reconstruction facility, wherein the reconstruction parameters of the second parameter set are used.

It may be provided here that the determination steps are carried out at least when fulfilling a further processing condition that evaluates the analysis information. It is conceivable, for example, that analysis information is determined which indicates that the preliminary image data set already fulfills the necessary requirements so that it can be used directly as a medical image data set. In many specific embodiments of the method according to the invention, however, it will be provided that the preliminary parameter set is selected in such a manner that, although the analysis information enables further individualization or adaptation of the reconstruction parameters for the specific patient, a second reconstruction or at least further processing of the preliminary image data set is useful in any case. This will be discussed in more detail below.

According to one or more example embodiments, it is therefore proposed to first reconstruct a preliminary image data set using a preliminary parameter set of reconstruction parameters which is the same for all patients—at least for the examination type, for example “screening”—and which is then provided to an analysis algorithm on an analysis facility. In other words, the preliminary image data set is transmitted from the first reconstruction facility to the analysis facility via a suitable communication link. There, the preliminary image data set is analyzed by the analysis algorithm with the aim of an initial assessment of the image content, so that analysis information is obtained on the basis of which a decision can be made as to which type of reconstruction of the medical image data set is the best for the individual patient, i.e. which makes the relevant, displayed image content, features and structures best recognizable and assessable in the evaluation. Examples of relevant image content, features and structures and their description include material densities, for example a breast density, risk classifications (for example so-called “risk scores”) and object types identified as likely to be contained, such as microcalcifications, spiculated and/or round masses, and others.

A second parameter set of reconstruction parameters can thus be selected and/or compiled on the basis of the analysis information, which can be carried out both on the analysis facility and, preferably, on the first or further reconstruction facility. A further, second reconstruction facility can be useful, for example, if the first reconstruction facility, which is preferably part of the medical imaging facility, is kept simple, i.e. is designed in particular only for reconstruction according to the preliminary parameter set and/or has low computing power and/or low storage capacities.

Depending on where the second parameter set is determined, the second parameter set and/or the analysis information is transferred from the analysis facility to the first or the further reconstruction facility. This now uses the second parameter set of reconstruction parameters to determine the medical image data set individually customized to the patient, in particular via re-reconstruction. However, it is also conceivable to determine the medical image data set at least partially on the basis of the preliminary image data set, for example through additional filtering and/or other post-processing.

In general, it can therefore be said that the second parameter set and the preliminary parameter set are different.

In this case, the preliminary parameter set can be understood as a type of standard parameter set or default parameter set. In particular, it can be adapted to the requirements of the analysis algorithm with regard to the preliminary image data set, thus allowing the provision of a preliminary image data set optimized for the analysis.

In other words, the present invention provides a combination of a plurality of medical facilities comprising the medical imaging facility, the first reconstruction facility (and optionally the further reconstruction facility) and the analysis facility. The analysis facility, which may also be referred to as an image analysis facility, may for example be designed to provide computer aided diagnosis (CAD) and/or clinical decision support system (CDSS) and/or computer aided risk scoring (CARS). The analysis facility can be a stand-alone product, for example a computing facility with software installed on it, or a pure software product that can run on a corresponding host facility, for example on a server, a computing facility of a diagnostic workstation and the like. In particular, the analysis facility can also be integrated into the medical imaging facility.

According to one or more example embodiments, the combination of at least one reconstruction facility and one analysis facility is combined with a data exchange between these facilities in order to provide improved medical image data sets for evaluation, for example by radiologists, and documentation. By using the analysis information to determine the second parameter set and thus also the medical image data set, it is therefore possible to optimize the individual image content of an acquisition process. The use of analysis equipment even before the final evaluation, in particular diagnosis, and the adaptation and/or selection of image reconstruction and/or image processing according to the analysis information, i.e. the image content, represents a completely new approach compared to the prior art mentioned in the introduction.

The combination of two medical facilities, namely the at least one reconstruction facility and the analysis facility as well as the new data interface between them, creates additional value for users, for example clinics, hospitals, radiology practices and the like. The reconstruction or processing of the medical image data set can now be optimized for the individual patient depending on the image content, particularly with regard to initial indications of possible findings and/or depending on anatomical features. Only a small amount of data needs to be transferred and the data exchange and reconstruction up to the medical image data set can take place without human interaction. From the evaluator's point of view, the proportion of cases that need to be reworked and, in particular, re-reconstructed can be minimized.

The approach according to one or more example embodiments can be understood as the use of two independent facilities, namely the at least one reconstruction facility and the analysis facility, which are connected by a communication link, in order to achieve an iterative optimization of the image processing and thus a simplification of the diagnosis task for the evaluator.

It should also be noted at this point that a diagnosis is only created by the evaluator during the evaluation, i.e. in a downstream step. Information obtained as part of the procedure described here, for example the analysis information, merely provides the evaluator with supporting information.

As already mentioned, it may be provided that the first reconstruction facility is part of the imaging facility. The integration of the first reconstruction facility into the medical imaging facility has the advantage that the preliminary image data set can be output on a display facility of the medical imaging facility so that an operator, in particular a medical-technical assistant (MTA), can immediately assess the success of the acquisition process and, if this is not the case, can, for example, schedule a repeat of the examination.

As already mentioned, the analysis facility is realized independently, i.e. separately from the first reconstruction facility, and is connected to it via a communication link. The data interface, which enables the iterative optimization of the reconstruction and image processing, is ultimately created via the communication link and the data exchange between the at least one reconstruction facility and the analysis facility.

In general, the procedure described here can be applied to a variety of examination types and imaging modalities. For example, the medical imaging facility can be or comprise a magnetic resonance facility and/or a computer tomography facility and/or a tomosynthesis facility and/or a PET facility and/or an ultrasound facility.

However, its use is always particularly advantageous if, as already explained, little information is available about the expected image content. In particular, raw data from a screening examination can therefore be used. In screening examinations, for example regular preventive examinations, patients are examined without a specific physical reason, so that it is unclear whether, and if so, which, abnormalities are present or occur. The analysis by the analysis algorithm on the basis of a standardized reconstruction using the preliminary parameter set allows an automated initial assessment, in particular the determination of possible abnormalities and/or anatomical features, whereupon a suitable second parameter set can be determined for the actual determination of the medical image data set, which provides a significantly better basis for assessment during evaluation by the evaluator.

In specific, preferred exemplary embodiments, it may be provided that the screening examination is a tomosynthesis examination of a female breast (mamma). An advantageous field of application for the present invention is therefore in mammography, for example in the context of breast cancer screening or the like. A large number of different abnormalities can occur here, for example calcifications, various tissue changes and the like, for which a wide variety of reconstruction and processing techniques can be useful. Despite the fact that in such screening patients are usually examined without a specific reason and without specific knowledge of possible abnormalities and anatomical features, one or more example embodiments makes it possible, via the analysis by the analysis facility and the corresponding feedback to the at least one reconstruction facility, to provide medical image data sets that are optimally suited to the individual patient for diagnosis by an evaluator. Even if specific examples below mainly relate to this preferred application example, one or more example embodiments is nevertheless not limited to this.

Preferably, an overview image data set, in particular a two-dimensional overview image data set, can be reconstructed as a preliminary image data set. Particularly in the case of three-dimensional imaging techniques, such as computer tomography and tomosynthesis, it can be useful to first reconstruct a two-dimensional overview image data set, from which basic abnormalities or their absence can already be seen. From such a two-dimensional overview image data set, an operator can also easily see whether the acquisition process was successful when displayed on a display facility of the medical imaging equipment.

Particularly preferably, the overview image data set may be a synthetic 2D image and/or one that corresponds to a predetermined acquisition process. For example, such a synthetic 2D image can be a projection image or correspond to such an image. For example, the synthetic 2D image can correspond to a fluoroscopic image in the X-ray field, while MIPs (maximum intensity projections) can also be used in other imaging techniques.

In the case of tomosynthesis, in particular mammography screening, the preliminary image data set can preferably be a synthetic 2D image, while the medical image data set is preferably a digital breast tomosynthesis image data set (DBT image data set). The synthetic 2D image can, for example, correspond to a fluoroscopic image taken during a conventional, two-dimensional X-ray breast examination, on which significant abnormalities and/or anatomical features may already be recognizable. The three-dimensional acquisition technique of tomosynthesis is then fully utilized for more precise evaluation, i.e. diagnosis, by generating a DBT image data set having multiple layers.

It may be provided that the analysis algorithm includes a trained analysis function. Particularly in the field of CAD, CDSS and CARS, there are already many approaches in the prior art for using artificial intelligence or machine learning. Neural networks, in particular convolutional neural networks (CNN), are particularly suitable for image evaluation, meaning that the trained analysis function can comprise a CNN in particular. However, “classic” sub-algorithms that do not require artificial intelligence can also be used. In particular, the analysis algorithm can comprise a combination of multiple trained analysis functions and/or multiple classic sub-algorithms. Suitable software packages that implement such analysis algorithms have already been proposed in the prior art.

Particularly advantageously, the analysis algorithm can be optimized and/or trained for the use of the preliminary parameter set. This therefore utilizes the fact that only preliminary image data sets are provided as input data, which are comparable due to the respective use of the preliminary parameter set. This makes it possible to program the analysis algorithm with regard to certain expectations, which can allow a simpler implementation. It is also conceivable to simplify machine learning when using a trained analysis function, in particular to reduce the amount of training data and/or variation to be learnt. More robust training results are also obtained. In other words, the analysis algorithm only has to adapt to one image style in terms of image properties and/or flavor. Overall, more consistent and reproducible analysis results are thus possible.

In specific embodiments, it may be provided that at least one annotation and/or a structured report, in particular in a predefined format, is determined as the analysis information. Both annotations and structured reports, in particular in a predefined format, can be easily evaluated automatically by a computing facility in order to determine the second parameter set.

For example, it may be provided that the analysis information comprises at least one score, in particular a risk score, and/or lesion information relating to at least one potential lesion. With regard to tomosynthesis of the breast, for example, corresponding analysis algorithms are already known, which can also be used in the context of the present invention and can provide a risk score and/or lesion information, for example relating to potential tumors and/or calcifications. Alternatively and/or additionally, the analysis information may also comprise a classification result, a segmentation result, a breast density classification and the like.

The reconstruction parameters that can be selected or adapted can ultimately comprise all conceivable, conventional reconstructions and/or modifications, for example filtering, contrast enhancement, binning, slice collapsing (in particular in DBT), denoising and the like. A higher dimensionality can also be provided for the medical image data set, as shown in the example above for the two-dimensional overview image in contrast to a three-dimensional reconstruction for the medical image data set. In particular, it may be provided that, compared to the preliminary parameter set, the reconstruction parameters of the second parameter set bring about a higher dimensionality and/or a higher resolution and/or a changed contrast and/or a contrast enhancement and/or a changed filtering and/or a changed binning and/or a changed and/or additional denoising and/or a changed layering property of layers of the reconstruction result and/or lead to a reconstruction of additional image data, wherein in particular the medical image data set is determined by supplementing the preliminary image data set with additional data. It is therefore also possible to provide additional data for the medical image data set, in particular in addition to or as an alternative to modified calculated image data. If, for example, DBT slices with a relatively high slice thickness are reconstructed for the preliminary image data set, very thin slices for indicated abnormalities, such as micro-calcifications or similar, can also be added for the medical image data set.

Generally and specifically, with regard to the second parameter set, it can be provided, for example, that
in the case of analysis information indicating a localized abnormality, in particular a potential lesion, a higher resolution and/or a reconstruction of additional data is selected at least locally around the abnormality,
in the case of a material-related abnormality, in particular one indicated by a score, the contrast with respect to the material and/or a tissue containing the material is increased,
a standard diagnostic parameter set is used as a second parameter set for analysis information that does not indicate an abnormality, and/or
the sharpness of the medical image data set is increased in the case of analysis information indicating potential calcifications.

In particular, it is even conceivable that reconstruction parameters of the second parameter set and/or the entire second parameter set are assigned to partial information or the entire analysis information in a look-up table. At least one such look-up table can, for example, be provided specifically for certain types of examination, such as mammography screening.

For archiving, the medical image data set can be stored in an image archiving facility, in particular for retrieval from a diagnostic facility of the person analyzing the image. Such image archiving facilities are already known in the prior art, for example as PACS (“picture archiving and communication system”). The medical image data sets can be retrieved from the image archiving facility, for example to a diagnostic facility, which may be a workstation computer at a diagnostic workstation, where the evaluation by the evaluator (i.e. the diagnosis) can take place.

In this context, the analysis information may also be stored in the image archiving facility. In this manner, the evaluator can be given further information in addition to the medical image data set, which makes it easier to evaluate the medical image data set. Documentation is also improved. All this is possible without massive additional storage effort, since only the analysis information, which usually has a significantly smaller storage size, is added. However, embodiments are also conceivable in which the preliminary image data set can also be stored in the image archiving facility for documentation purposes.

Preferably, the medical image data set can be evaluated by the analysis facility using an evaluation algorithm to determine evaluation information that describes the image content. The evaluation by the evaluator can also be supported by computer-aided evaluation, which can be provided as evaluation information by the analysis facility. Here too, the classic CAD, CDSS and CARS approaches already mentioned should be mentioned. The evaluation information can also be stored appropriately in the image archiving facility. In any case, it is provided for information purposes with the medical image data set. If the analysis information is understood as the first analysis information, the evaluation information can also be referred to as the second analysis information.

In a particularly preferred further development, it may be provided that at least part of the communication, in particular the specification of a further processing target and/or a task to be carried out, is carried out using the DICOM standard and/or at least the first reconstruction facility, in particular also the further reconstruction facility, and the analysis facility are designed as DICOM clients. The properties of the DICOM standard, which are advantageously available in any case, can therefore also be used in the context of the present invention beyond the pure format of the image data sets, so that the DICOM standard can be used, for example, to specify the target, in particular the DICOM client, for which an image data set is intended and which measures are to be carried out there. In this embodiment, the DICOM standard is therefore used to specifically design the data interface between the at least one reconstruction facility and the analysis facility.

Expediently, it can be provided that an analysis result, which is determined by evaluating at least one user input from the evaluator, is archived with the medical image data set. In this manner, the documentation, in particular on the image archiving facility, is also supplemented and thus completed by the evaluation result of the evaluator. For example, a radiologist or the like can perform a diagnosis on the medical image data set as an evaluator and, for example, enter diagnosis results, in particular diagnoses performed by the evaluator outside the process sequence described here, as an evaluation result, which can then be stored in association with the medical image data set.

In addition to the method, the present invention also relates to a provision system for a medical image data set, comprising a medical imaging facility for acquiring raw data in an acquisition process, at least one reconstruction facility and an analysis facility, wherein the at least one reconstruction facility and the analysis facility are connected via a communication link, in particular for establishing a data interface. According to one or more example embodiments, it is provided here that
a first reconstruction facility of the at least one reconstruction facility is designed to reconstruct a preliminary image data set from the raw data, wherein reconstruction parameters of a predetermined default parameter set are used,
the analysis facility is designed to analyze the preliminary image data set via an analysis algorithm to determine an item of analysis information that describes the image content,
the first or a further reconstruction facility of the at least one reconstruction facility or the analysis facility is designed to determine a second parameter set of reconstruction parameters as a function of the analysis information, and
the first or the further reconstruction facility is designed to determine the medical image data set from the raw data and/or the preliminary image data set, wherein the reconstruction parameters of the second parameter set are used.

All explanations relating to the method according to one or more example embodiments can be transferred analogously to the provision system according to one or more example embodiments and vice versa, so that the advantages already mentioned can therefore also be achieved with the latter, in particular patients can be provided with individually, preferably optimally, reconstructed medical image data sets which are prepared in relation to the image content.

In particular, for the provision system it also applies that the first reconstruction facility is preferably implemented as part of the medical imaging facility. In this sense, in corresponding exemplary embodiments, the preliminary image data set is also reconstructed directly on the medical imaging facility and can also be displayed there to an MTA to check the success of the acquisition. The medical imaging facility may in particular be a tomosynthesis facility for examining the female breast, in particular for mammography, on which screening examinations are performed.

A computer program product according to one or more example embodiments can be loaded directly into at least one storage means (e.g., a computer-readable storage medium) of a provision system and has program means (e.g., program code) such that, when the computer program product is executed on the provision system, the latter is caused to carry out the steps of a method according to one or more example embodiments. The computer program product can be stored on an electronically readable data carrier, i.e. a computer-readable storage medium, which thus comprises control information stored thereon, which comprises at least one computer program product according to one or more example embodiments and is designed in such a manner that, when the data carrier is used in a provision system, it causes the latter to execute a method according to one or more example embodiments. In particular, the data carrier can be a non-transient data carrier, such as a CD-ROM.

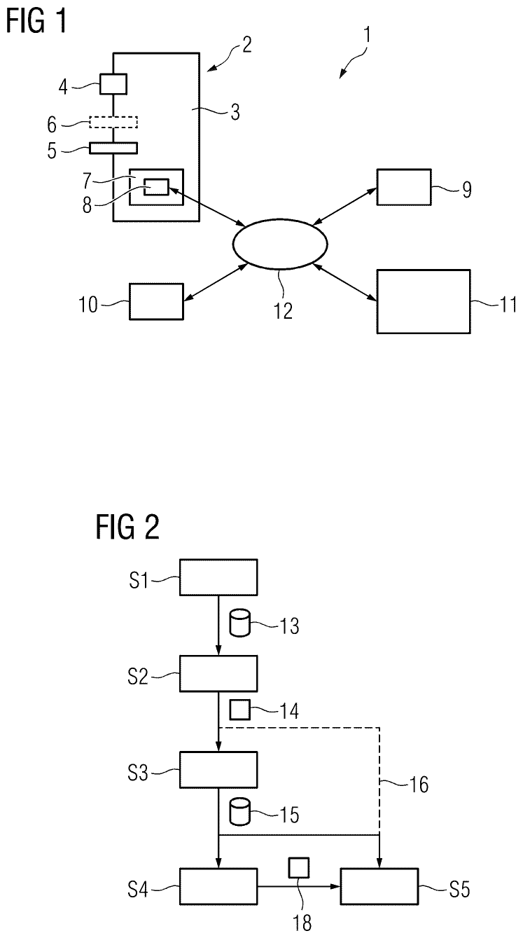

FIG. 1 shows a first exemplary embodiment of a provision system 1 according to the invention. This comprises a medical imaging facility 2, which in the present case is shown as a tomosynthesis facility 3 for use in mammography. The imaging facility 2 thus comprises in the exemplary embodiment in addition to an X-ray emitter 4, which can be moved for tomosynthesis, and an X-ray detector 5 also a compression plate 6. In the context of the present invention, other imaging facilities 2 are also conceivable in other exemplary embodiments, for example a computer tomography facility, a magnetic resonance facility and/or a PET facility.

The operation of the imaging facility 2 is controlled by a control unit 7, which in the present case also comprises a reconstruction facility 8 (first reconstruction facility 8). The provision system 1 comprises moreover an analysis facility 9, a diagnostic computer 10 at a diagnostic work station and an image archiving facility 11, in the present case a PACS system. The reconstruction facility 8, the analysis facility 9, the diagnostic computer 10 and the image archiving facility 11 are connected via a network 12, so that communication links can be established between these components of the provision system 1. The analysis facility 9 can also be provided as part of the medical imaging facility 2 as a medical facility separate from the reconstruction facility 8. At least the reconstruction facility 8 and the analysis facility 9 are designed as DICOM clients, so that they can in particular evaluate DICOM metadata with regard to an addressee and a processing task to be performed.

The provision system 1 is designed to perform a first exemplary embodiment of the method according to the invention, which will now be explained in more detail with reference to the provision system 1 and FIG. 2, further exemplified specifically for mammography screening examinations.

After the acquisition of raw data, in this case projection images of a patient's breast, this raw data is used in a step S1 to reconstruct a preliminary image data set 13 in the first reconstruction facility 8 in accordance with a predetermined set of default parameters, which is specified for all screening examinations of this type. In the present specific example of breast tomosynthesis, the preliminary image data set 13 is a two-dimensional overview image, in particular a synthetic 2D image.

The preliminary image data set 13 is now transmitted in DICOM format via a communication link established via the network 12 to the analysis facility 9, which can determine, for example on the basis of the DICOM metadata, that the preliminary image data set 13 is to be analyzed in a step S2 according to an analysis algorithm in order to determine an item of analysis information 14. The analysis information 14 can, for example, contain at least one score and/or at least one annotation and, generally speaking, indicate potential abnormalities and/or anatomical features. In order to determine the analysis information 14, the analysis algorithm can, for example, comprise a trained analysis function that has been trained by machine learning. In any case, the analysis algorithm is developed specifically for image data sets that are determined in advance in accordance with the default parameter set and optimized for these, in particular also trained. The analysis information can also usefully comprise a structured report, but in any case can be designed overall for machine-based evaluation.

In the present exemplary embodiment, the analysis information 14 is now transmitted back to the reconstruction facility 8, which evaluates the analysis information in a step S3 and, based on this, determines a second parameter set with reconstruction parameters, which are also used in step S3, in order to reconstruction a medical image data set 15 by the reconstruction facility 8. It should be noted that the second parameter set can also be determined on the basis of the analysis information 14 on the analysis facility 9, which then transmits the second parameter set to the reconstruction facility 8 accordingly. In turn, all transmissions take place via the communication link established via the network 12.

In the present, specific exemplary embodiment of breast tomosynthesis, the medical image data set 15 is reconstructed as a three-dimensional DBT image data set, i.e. determined with a higher dimensionality than the preliminary image data set 13. In general, depending on the analysis information 14, it may be provided that certain abnormalities in the medical image data set 15 are emphasized or contrasts are selected accordingly, a higher resolution is provided at least in the area of abnormalities and/or anatomical features, filtering and/or other image processing is performed that is adapted to the results of the analysis according to the analysis information 14, additional data is determined and the like. In other words, the analysis information 14 is used to provide a medical image data set 15 optimized for evaluation by an evaluator.

Accordingly, the medical image data set 15 is provided to the image archiving facility 11 in step S5. In the present case, however, it is also transmitted again to the analysis facility 9, where an evaluation algorithm is applied to it in step S4 in order to provide evaluation information 18, which is then also stored in the image archiving facility 11, assigned to the medical data set 15. The evaluation algorithm may at least partially use components of the analysis algorithm and/or be comparable to it. The evaluation information 18 is to be used to support the evaluation (“decision support”).

As indicated by the dashed arrow 16, the analysis information 14, assigned to the medical image data set 15, can optionally also be stored in the image archiving facility 11.

The medical image data set 15, together with the analysis information 14 and the evaluation information 18, can then be retrieved from the image archiving facility 11 to the diagnostic computer 10. An evaluator analyzing the medical image data set 15 can enter their evaluation result, for example a diagnosis, on the diagnosis computer 10 so that this can also be archived with the medical image data set 15 on the image archiving facility 11.

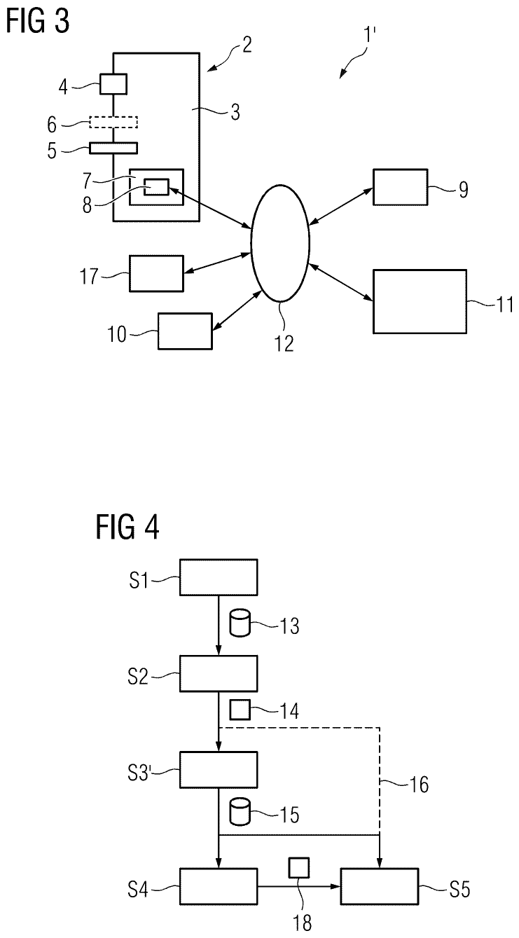

FIG. 3 shows a slightly modified exemplary embodiment of a provision system 1′ compared to FIG. 1, in which the same reference characters stand for the same components. In contrast to FIG. 1, in this case a second reconstruction facility 17 is used in addition to the first reconstruction facility 8. As the modified exemplary embodiment of the method according to the invention as shown in FIG. 4 indicates, the analysis information 14 is not sent to the first reconstruction facility 8 after determination in step S2 on the analysis facility 9, but rather is sent to the second reconstruction facility 17, where, in the modified step S3′, the medical image data set 15 is determined and forwarded accordingly. Accordingly, the raw data is provided to the further reconstruction facility 17 by the medical imaging facility 2 via the network 12.

The use of a further reconstruction facility 17 may be appropriate if a simpler first reconstruction facility 8 is to be implemented on the medical imaging facility 2, which is suitable for the (possibly simpler) reconstruction of the preliminary image data set 13, although the further reconstruction facility 17, which for example has greater computing power, offers further possibilities for the possibly more complex reconstruction of the medical image data set 15 in accordance with the second parameter set.

Although the invention has been further illustrated and described in detail with the aid of example embodiments, the invention is not limited by the disclosed examples and other variations can be derived therefrom by the person skilled in the art without departing from the protective scope of the invention.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage 1 medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash a drive, memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for determining a medical image data set from raw data of an acquisition process using a medical imaging facility, the method comprising:
reconstructing a preliminary image data set from the raw data via a first reconstruction facility, wherein reconstruction parameters of a predetermined default parameter set are used;
analyzing the preliminary image data set via an analysis algorithm on an analysis facility to determine an item of analysis information that describes image content of the preliminary image data set;
determining a second parameter set of reconstruction parameters as a function of the analysis information; and
determining the medical image data set from at least one of the raw data or the preliminary image data set via the first reconstruction facility or a further reconstruction facility, wherein the reconstruction parameters of the second parameter set are used to determine the medical image data set.

2. The method of claim 1, wherein raw data from a screening examination is used.

3. The method of claim 2, wherein the screening examination is a tomosynthesis examination of a female breast.

4. The method of claim 1, wherein an overview image data set is reconstructed as the preliminary image data set.

5. The method of claim 1, wherein at least one of,
the analysis algorithm comprises a trained analysis function
the analysis algorithm is optimized, or
the analysis algorithm is trained for use on the predetermined default parameter set.

6. The method of claim 1, wherein at least one of at least one annotation or a structured report is determined as the analysis information.

7. The method of claim 1, wherein the analysis information comprises at least one score, in particular a risk score, and/or lesion information relating to at least one potential lesion.

8. The method of claim 1, wherein compared to the predetermined default parameter set, the reconstruction parameters of the second parameter set bring about at least one of a higher dimensionality, a higher resolution, a changed contrast, a contrast enhancement, a changed filtering, a changed binning, a changed, additional denoising, a changed layering property of layers of from the reconstructing, or lead to a reconstruction of additional image data, wherein in particular the medical image data set is determined by supplementing the preliminary image data set with additional data.

9. The method of claim 1, wherein the medical image data set is stored in an image archiving facility.

10. The method of claim 1, wherein the medical image data set is evaluated by the analysis facility using an evaluation algorithm to determine evaluation information that describes the image content.

11. The method of claim 1, wherein at least one of a further processing target or a task to be carried out is carried out using DICOM.

12. The method of claim 1, wherein an evaluation result, which is determined by evaluating at least one user input from an evaluator, is archived with the medical image data set.

13. A provision system for a medical image data set, comprising:
a medical imaging facility configured to acquire raw data in an acquisition process;
at least one reconstruction facility; and
an analysis facility, wherein the at least one reconstruction facility and the analysis facility are connected via a communication link, wherein
a first reconstruction facility of the at least one reconstruction facility is configured to reconstruct a preliminary image data set from the raw data, wherein reconstruction parameters of a predetermined default parameter set are used,
the analysis facility is configured to analyze the preliminary image data set via an analysis algorithm to determine an item of analysis information that describes image content of the preliminary image data set,
the first reconstruction facility, a further reconstruction facility or the analysis facility is configured to determine a second parameter set of reconstruction parameters as a function of the analysis information, and
the first reconstruction facility or the further reconstruction facility is configured to determine the medical image data set from at least one of the raw data or the preliminary image data set, wherein the reconstruction parameters of the second parameter set are used to determine the medical image data set.

14. The provision system of claim 13, wherein the first reconstruction facility is part of the medical imaging facility.

15. A non-transitory computer program product including instructions which, when executed in a provision system, causes the provision system to perform the method of claim 1.

16. The method of claim 4, wherein the overview image data set is two-dimensional.

17. The method of claim 6, wherein a structured report in a predefined format is determined as the analysis information.

18. The method of claim 8, wherein in particular the medical image data set is determined by supplementing the preliminary image data set with additional data.

19. The method of claim 9, wherein the medical image data set is stored in the image archiving facility for retrieval from a diagnostic facility of an evaluator.

20. The method of claim 1, wherein at least the first reconstruction facility and the analysis facility are designed as DICOM clients.
